(19) United States
(12) Plant Patent Application Publication
(10) Pub. No.: US 2004/0025215 P1
(43) Pub. Date: Feb. 5, 2004

(54) Bunashimeji mushroom plant named Hokuto Shiro Ichigoukin

(75) Inventors: Makoto Ishii, Nagano-shi (JP); Hisae Imai, Nagano-shi (JP); Mizue Yamazaki, Nagano-shi (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: HOKUTO SANGYO KABUSHIKI KAISHA, Nagano-shi (JP)

(21) Appl. No.: 10/624,520

(22) Filed: Jul. 23, 2003

(30) Foreign Application Priority Data
Jul. 23, 2002 (JP) 14858

(51) Int. Cl. A01H 15/00

(52) U.S. Cl. PLT/394

(57) ABSTRACT

The present variety of mushroom plant named Hokuto Shiro Ichigoukin was produced by crossbreeding mutant fungal strains of two parent varieties, a female variety Hokuto Hachigoukin and a male variety Hokuto Jyunigoukin, after UV irradiation. This edible mushroom is not bitter, and can be cultivated anywhere a cultivation facility can be arranged.

The present plant patent application claims priority of Japanese plant variety application No. 14858, filed Jul. 23, 2002, which is hereby incorporated by reference.

Hokuto Shiro Ichigoukin is a variety of Bunashimeji mushroom plant that was produced by improving the existing varieties to have the following properties: nice white color, no bitter taste, delicious taste with a higher saccharide content, and cultivatable under similar conditions, including cultivation cycle. The scientific name of the variety is Hypsizygus marumoreus. The present plant is an edible mushroom.

### BACKGROUND OF THE INVENTION

The present variety Hokuto Shiro Ichigoukin was produced using mutant fungal strains of two parent varieties, Hokuto Hachigoukin and Hokuto Jyunigoukin, grown by Hokuto Sangyo Kabushiki Kaisha. Specifically, the female variety was Hokuto Hachigoukin and the male variety was Hokuto Jyunigoukin. Hokuto Hachigoukin and Hokuto Jyunigoukin were each submitted to UV irradiation, and then the mutated varieties were crossbred to produce the present variety Hokuto Shiro Ichigoukin.

The site of development was on property of Hokuto Sangyo Kabushiki Kaisha Kinoko Sougo Kenkyusho in Nagano, Japan.

In April 2000, the mutated Hokuto Hachigoukin was produced, in that a UV-irradiated strain was spawned on a culture medium. In August 2000, the mutated Hokuto Hachigoukin was harvested, and monospores were isolated.

In parallel, in May 2000, the mutated Hokuto Jyunigoukin was produced, also in that a UV-irradiated strain was spawned on a culture medium. In October 2000, the mutated Hokuto Jyunigoukin was harvested, and monospores were isolated.

In September and October 2000, primary hyphae were obtained from mutated Hokuto Hachigoukin and mutated Hokuto Jyunigoukin. From December 2000, mutated Hokuto Hachigoukin and mutated Hokuto Jyunigoukin were used to obtain crossbred stains. In February 2001, cultivation was started, and harvesting began in May 2001.

Further, in June 2001, a cultivation test was started for secondary selection. In October 2001, an HM222 strain, which appeared to be a superior variety, was selected. In December 2001, a cultivation test was initiated for stabilization.

In January 2002, a characteristic test was started. In May 2002, after repeated cultivation tests, stability, reproducibility and homogeneity, matching the objectives of improvements, were confirmed, i.e., the asexually propagated clones of the present plant are identical to the original plant in all distinguishing characteristics. Thus the HM222 strain was named Hokuto Shiro Ichigoukin.

### SUMMARY OF THE INVENTION

The present invention provides a variety, Hokuto Shiro Ichigoukin, which is an edible mushroom. When this mushroom is cooked in hot water, the bitterness associated with bunashimeji is mostly gone, thus making it a delectable variety. The present variety can be cultivated anywhere a cultivation facility can be arranged.

In summary, the morphological characteristics of Hokuto Shiro Ichigoukin are as follows: at the appropriate harvesting period, the cap is small and round; the color of the cap center and margin is white; the thickness and quality of the cap meat are medium; the number of mottled spots is medium; the mottled spots are distributed in the central area; the clarity of the mottled spots is medium; the color of the gills is yellowish white; the arrangement of the gills is orderly; the width and density of the gills are medium; the length of the stipe is medium; attachment of the stipe to the cap is centric; the shape of the stipe is moderately thick; the color of the stipe is white; there is no hair on the stipe; and the quality of the stipe meat is medium.

As to the cultivation data for Hokuto Shiro Ichigoukin, yield is about 118.5 g, the number of effective fruit bodies is 62, and the length of time from spawning to harvesting is 91 days.

### BRIEF DESCRIPTION OF THE DRAWINGS

FIG. 1 is a photograph showing a front view of the present plant variety cultivated in vases;

FIG. 2 is a photograph showing a front view of the present variety without growth substrate;

FIG. 3 is a photograph showing a perspective view of the present plant variety cultivated in vases;

FIGS. 4a and 4b are photographs showing a surface and a back, respectively, of a culture of the present plant variety;

FIG. 5 is a photograph showing a front view of a parent plant variety, Hokuto Hachigoukin, cultivated in vases;

FIGS. 6A and 6B are photographs showing a surface and a back, respectively, of a culture of parent plant variety Hokuto Hachigoukin;

FIG. 7 is a photograph showing a front view of another parent plant variety, Miyuki M-8171, cultivated in vases; and

FIGS. 8A and 8B are photographs showing a surface and a back, respectively, of a culture of parent plant variety Miyuki M-8171.

FIGS. 9A and 9B are a table and a graph, respectively, showing comparative mycelial growth of the present plant variety, as compared with that of parent plant varieties Hokuto Hachigoukin and Miyuki M-8171.

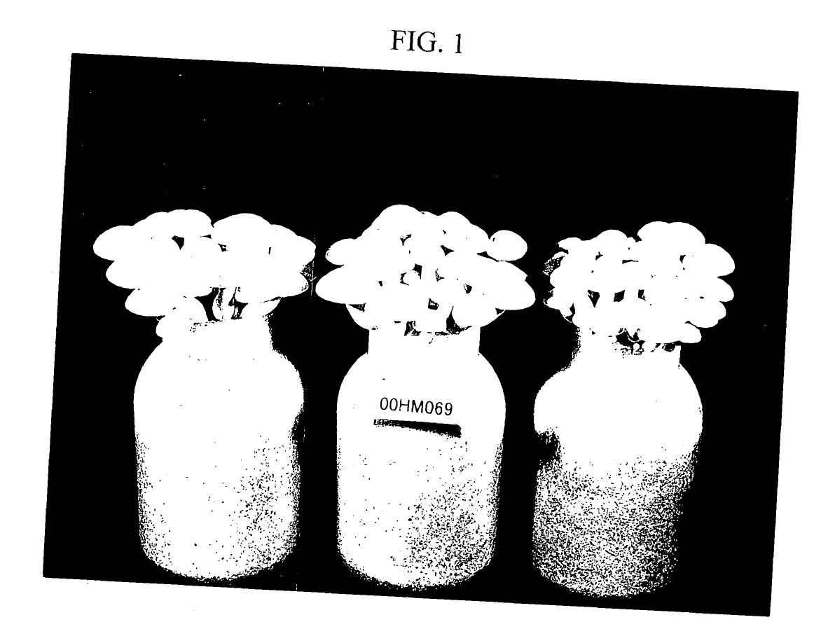

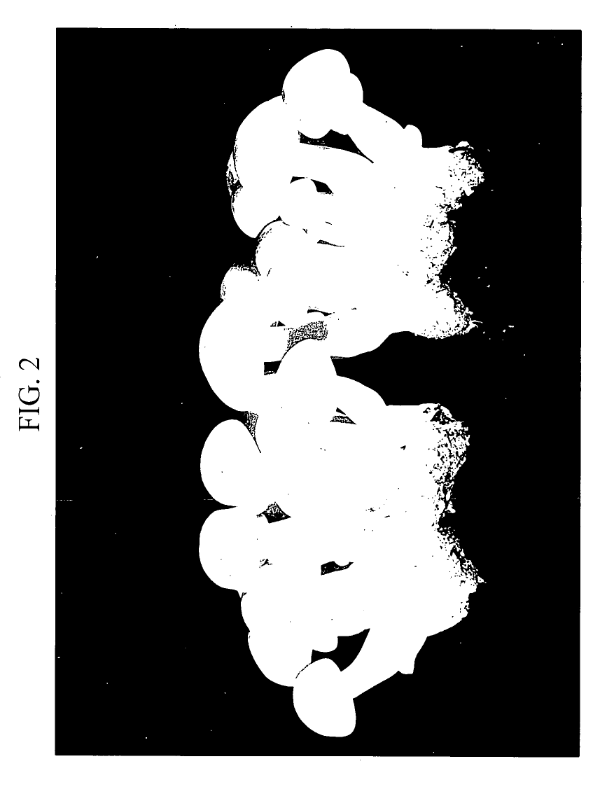

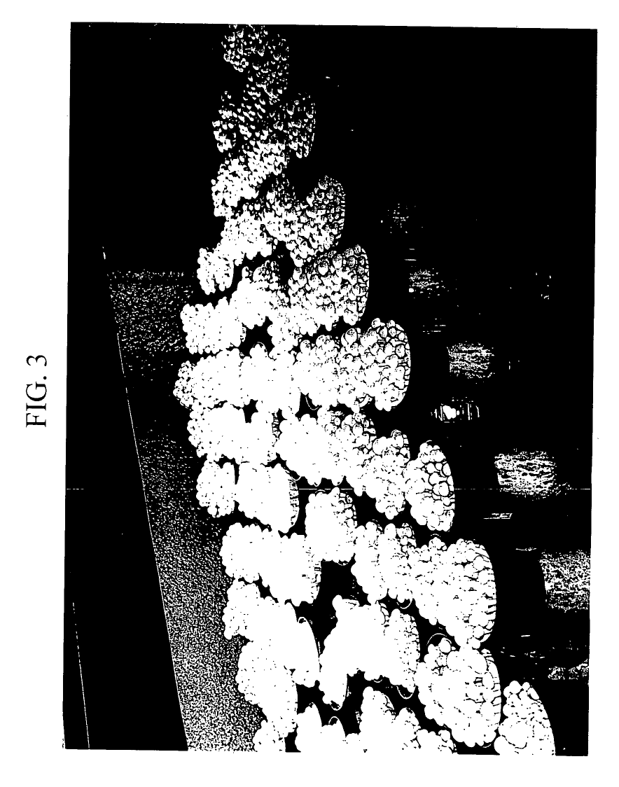

### DETAILED DESCRIPTION OF THE INVENTION

The characteristics of the present variety will be described in comparison with a corresponding variety, Hokuto Hachigoukin, and with another corresponding variety, Miyuki M-8171, which is commercially available (samples were bought at a supermarket in Nagano, Japan).

The cultivation site for this comparison was Hokuto Sangyo Kabushiki Kaisha in Nagano, Japan. The cultivation period was January to June 2002. The cultivation was made according to the conventional methods in a facility with a culture room of approximately 60 m³ in size and a growth room of approximately 30 m³ in size. In both the culture and growth rooms, an air-conditioning unit, comprising a cooler and heat exchanger, was installed, and in the growth room, cultivation racks and lights were placed. For characteristic analyses, three cultivation tests were performed using 25 bottles per block. The cultivation method used nutrient propagation, and strains were used as seeds.

In summary, when compared to Hokuto Hachigoukin, Hokuto Shiro Ichigoukin exhibits the following characteristics: formation of an inhibition zone in dual culture; dense aerial hyphae (Hachigoukin: medium density); cross sectional shape of hat is umbonate (Hachigoukin: umbonate to flat); white cap center (Hachigoukin: yellowish brown); white cap margin (Hachigoukin: light grayish brown); medium quantity of mottled spots (Hachigoukin: many); central distribution of mottled spots (Hachigoukin: all areas except margin); yellowish white colored gills (Hachigoukin: white); and medium length stipe (Hachigoukin: long).

When compared to the commercial variety, Hokuto Shiro Ichigoukin exhibits the following characteristics: formation of an inhibition zone in dual culture; dense aerial hyphae (commercial variety: medium density); clumped pattern for fruit body development (commercial variety: grouped pattern); central distribution of mottled spots (commercial variety: all areas except margin); yellowish white colored gills (commercial variety: white); a diameter under cap to maximum diameter ratio of 1.59 (commercial variety: 1.6-1.8); and a cap diameter to stripe length ratio of ≧2. 1 (commercial variety: 1.5-2.0).

The characteristics of the present plant variety will now be described in details, with relevant comparisons with varieties Hokuto Hachigoukin and Miyuki M-8171.

(1) Dual Culture

The test was done according to the examination standards established for Shirotamogitake (Hypsizygus ulmarius). With regard to the inhibition zone, the results were shown in Table 1 below (+: inhibition zone, −: no inhibition zone formation).

TABLE 1
Inhibition Zone

| | Hokuto Shiro | Hokuto | |
|---|---|---|---|
| Similar variety | Ichigoukin | Hachigoukin | Miyuki M-8171 |
| Hokuto Shiro | − | + | + |
| Ichigoukin | | | |

Thus, an inhibition zone was formed between Hokuto Shiro Ichigoukin and the comparative varieties Hokuto Hachigoukin as well as Miyuki M-8171, which is the closest variety after Hokuto Hachigoukin.

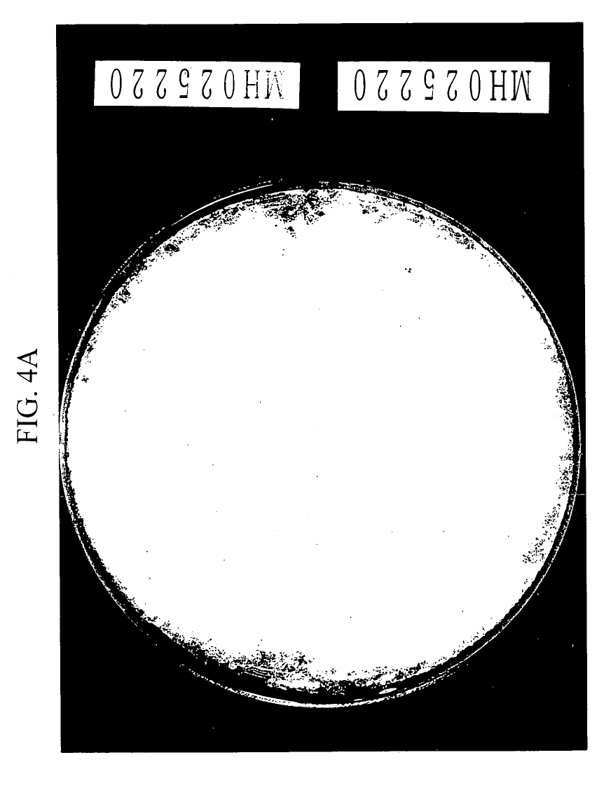

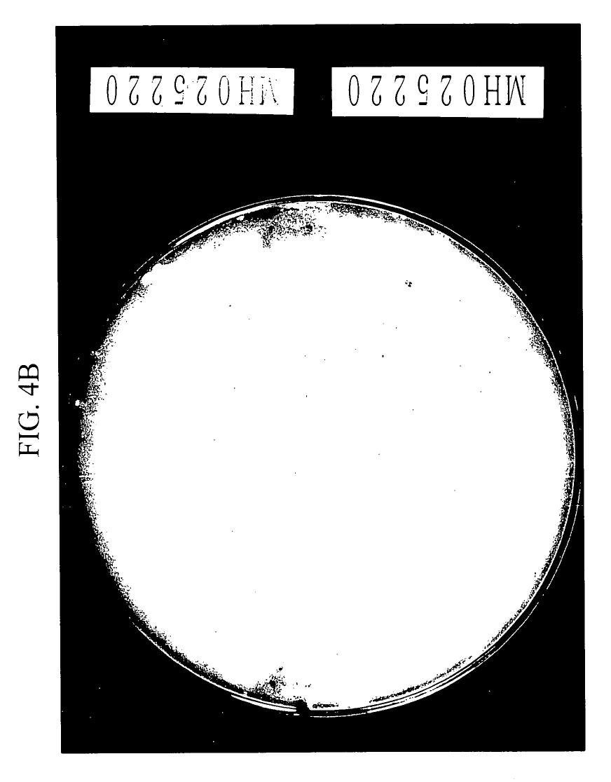

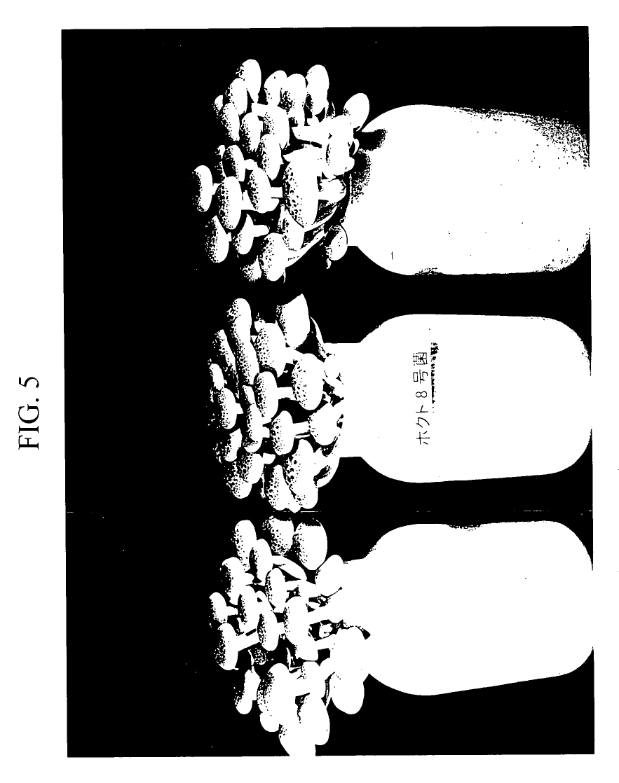

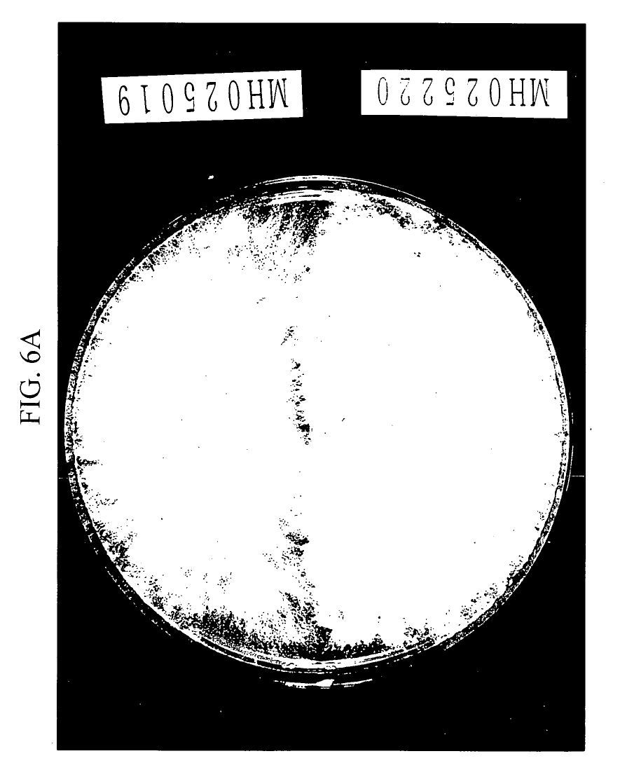

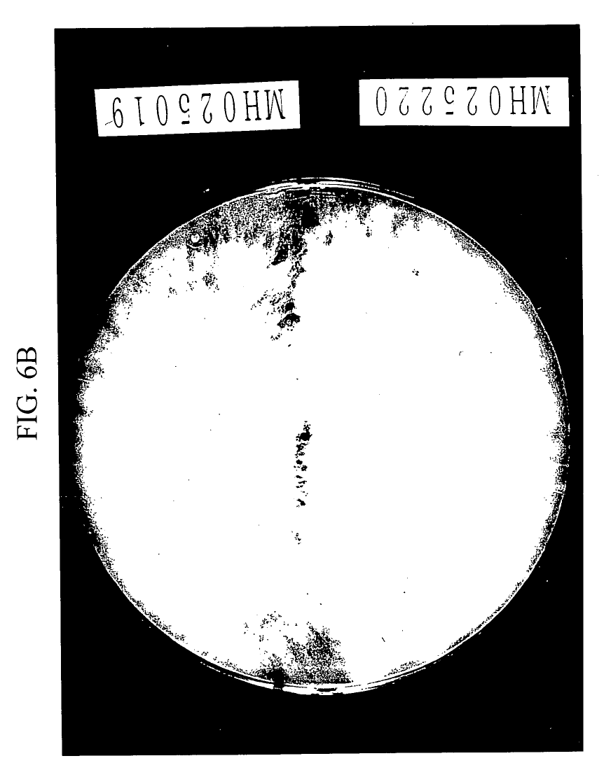

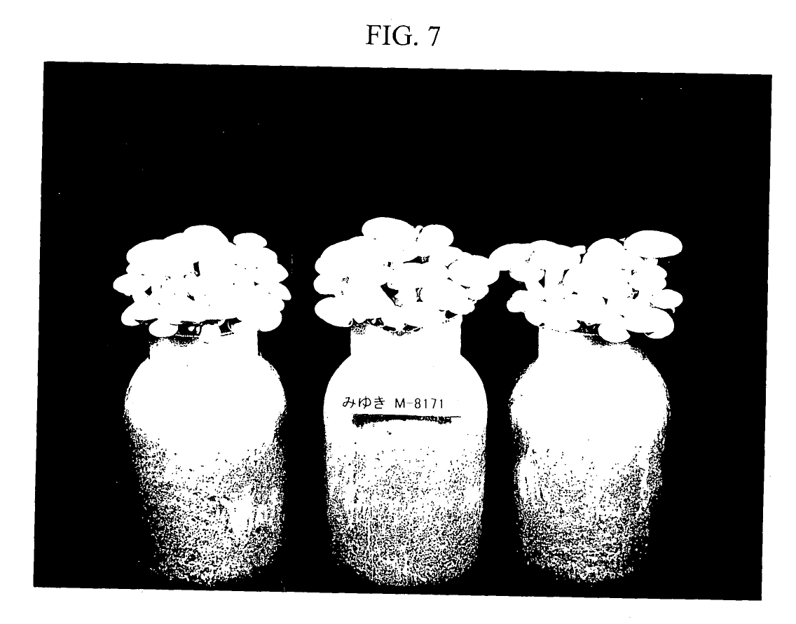

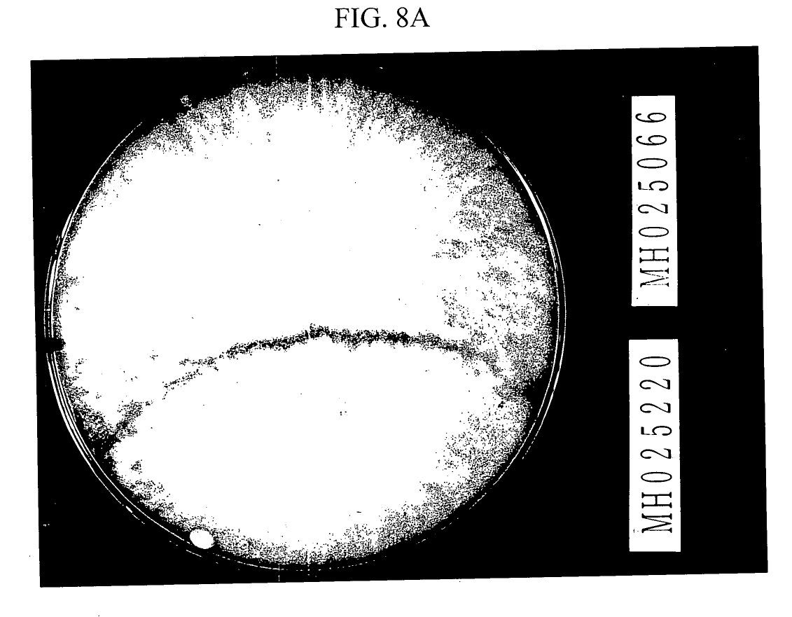

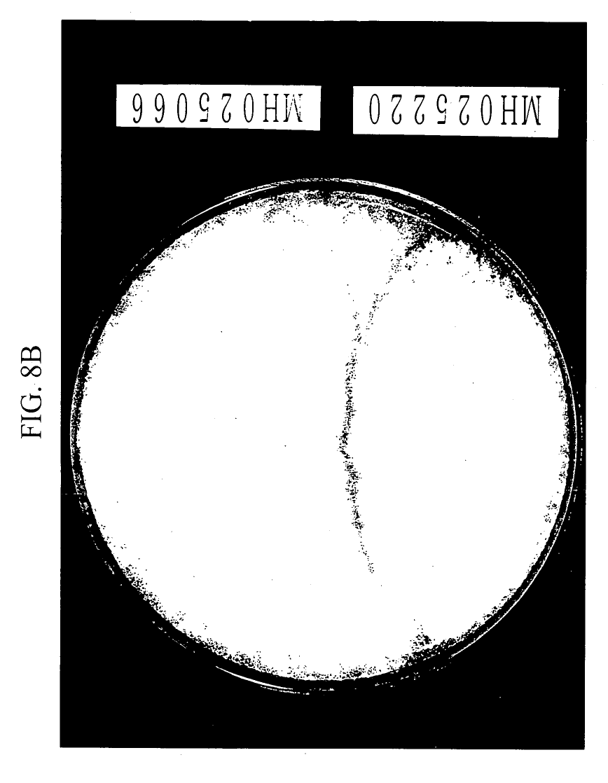

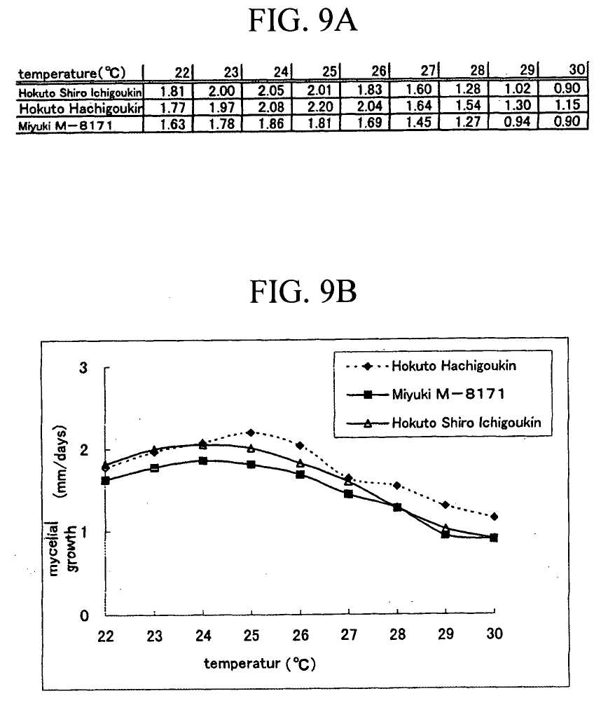

Photographs of the cultures for the present plant variety and for comparative varieties Hokuto Hachigoukin and Miyuki M-8171 are shown on FIGS. 4, 6 and 8, respectively.

(2) Length of Culture from Spawning to Fruit-Body-Formation Promotion

The length of culture from spawning to fruit-body-formation promotion was investigated. Again, the test period was January-June 2002. Except for the number of cultivation days, cultivation was done according to the examination standards established for Shirotamogitake (Hypsizygus ulmarius). Three lengths of culture were established at 63, 70 and 77 days. The results are summarized in Table 2 below.

TABLE 2
Culture Length

| Culture length (days) | Growth length (days) | Yield (g/bottle) |
|---|---|---|
| 63 | 20.5 | 128.0 |
| 70 | 20.5 | 143.4 |
| 77 | 22.0 | 115.8 |

Based on the results shown in Table 2, the length of culture was set at 70 days because the yield was the highest for this length.

(3) Optimal Budding Temperature

To determine the optimal budding temperature, cultivation was done for 70 days according to the examination standards established for Shirotamogitake (Hypsizygus ulmarius). Three budding temperatures were established as shown in Table 3 below.

TABLE 3
Optimal Budding Temperature

| Budding temperature (° C.) | Budding length (days) |
|---|---|
| 13˜14 | 6.7 |
| 14˜15 | 6.3 |
| 15˜16 | 6.9 |

Based on the results shown in Table 3, the minimal temperature was set at 14-15° C. because the length of budding was the shortest at this temperature.

(4) Optimal Growth Temperature

The optimal growth temperature was studied through cultivation for 70 days according to the examination standards established for Shirotamogitake (Hypsizygus ulmarius). Three growth temperatures were established as shown in Table 4 below.

TABLE 4
Growth Temperature

| Growth temperature (° C.) | Growth length (days) | Yield |
|---|---|---|
| 13˜14 | 22.1 | 115.6 |
| 14˜15 | 21.2 | 121.1 |
| 15˜16 | 24.9 | 107.1 |

Based on the results shown in Table 4, the optimal growth temperature was set at 14-15° C. because the yield was the greatest at this temperature.

(5) Summary of Cultivation Characteristics

The cultivation characteristics of the present plant variety are shown in Table 5 below.

TABLE 5
Summary of Cultivation Characteristics

| | Hokuto | | |
|---|---|---|---|
| | Shiro | Hokuto | Miyuki |
| Characteristics | Ichigoukin | Hachigoukin | M-8171 |
| Length of culture from spawning | 70 | 70 | 70 |
| to fruit-body-formation promo- | | | |
| tion (days) | | | |
| Length of time from fruit-body- | 21 | 21 | 21 |
| formation promotion to fruit | | | |
| body harvesting at optimal temp- | | | |
| erature (days) | | | |
| Optimal budding temperature | 14˜15 | 14˜15 | 14˜15 |
| (° C.) | | | |
| Fruit body development pattern | Clumped | Clumped | Group |
| Yield (g/bottle) | 118.5 | 117.1 | 105.1 |
| Number of effective fruit bodies | 62 | 100 | 79 |
| Medium compatibility | Sawdust | Sawdust | Sawdust |
| Tree selectivity | Mixture | Mixture | Mixture |
| Cap Size (mm) | 23.2 | 23.4 | 24.9 |
| Cross sectional shape | Umbonate | Umbonate | Umbonate |
| Center color | White | Yellowish | White |
| | | brown | |
| Margin color | White | Light grayish | White |
| | | brown | |
| Meat thickness (mm) | 8.1 | 6.8 | 7.4 |
| Meat quality | Medium | Medium | Medium |
| Mottled spot quantity | Medium | Many | Medium |
| Mottled spot size | Medium | Medium | Medium |
| Mottled spot distribution | Central | All areas | All areas |
| | area | except | except |
| | | margin | margin |
| Mottled spot clarity | Medium | Medium | Medium |
| Cracking | Absent | Absent | Absent |
| Gills Color | Yellowish | White | White |
| | white | | |
| Arrangement | Orderly | Orderly | Wavy and |
| | | | frizzled |
| Width | Medium | Medium | Medium |
| Density | Medium | Medium | Medium |
| Stipe | | | |
| Length (mm) | | | |
| Attachment to cap | Centric: | Centric: | Centric: |
| | eccentric | eccentric | eccentric |
| Shape | Medium | Medium | Medium |
| | thick | thick | thick |
| Color | White | White | White |
| Hair | Absent | Absent | Absent |
| Diameter under cap to maximum | | | |
| diameter ratio | | | |
| Cap diameter to stipe length | | | |
| ratio | | | |
| Meat quality | Medium | Medium | Medium |

(6) Cultivation Characteristic Data

Detailed cultivation characteristics of the present plant variety are shown in Table 6 below.

TABLE 6
Detailed Cultivation Characteristics

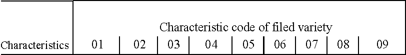

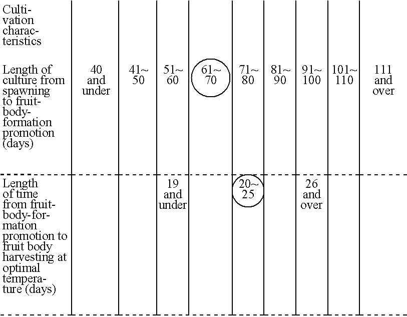

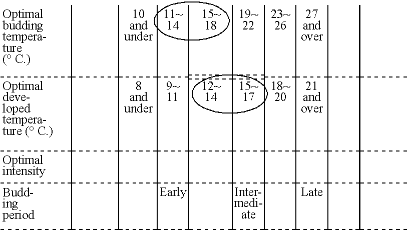

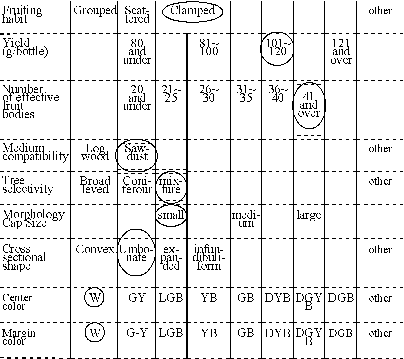

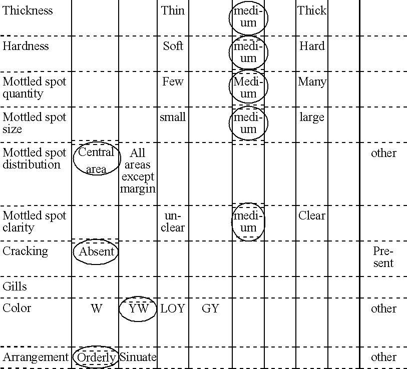

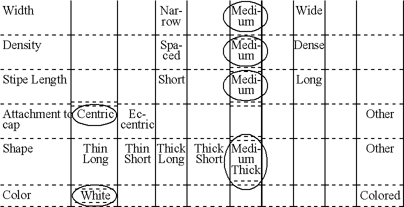

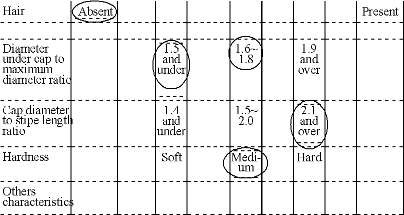

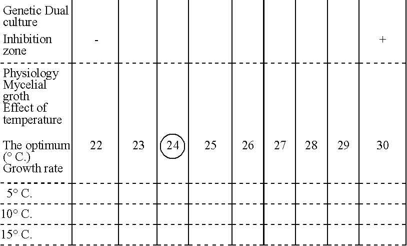

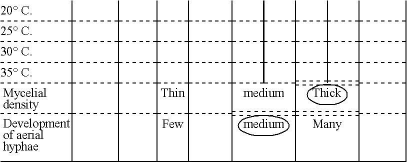

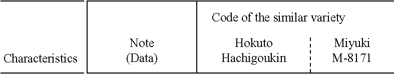

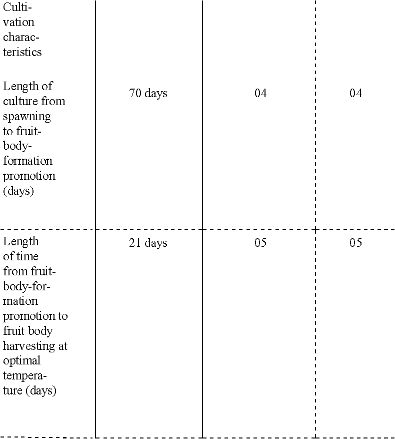

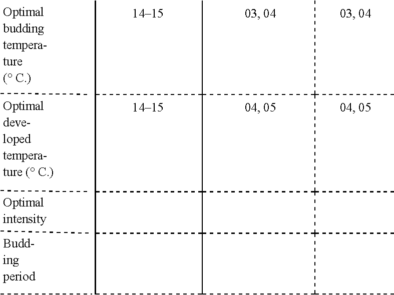

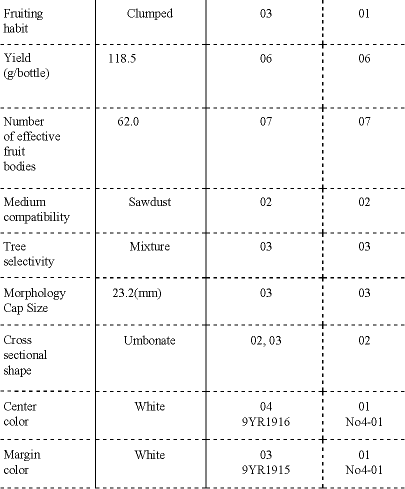

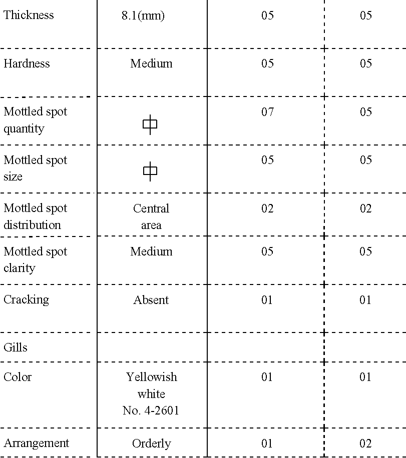

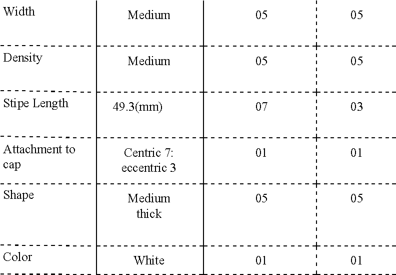

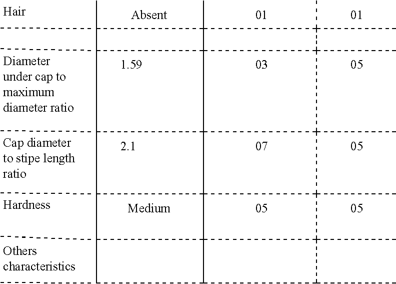

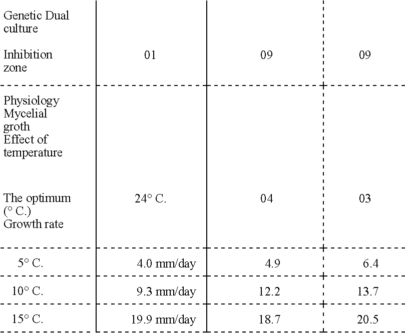

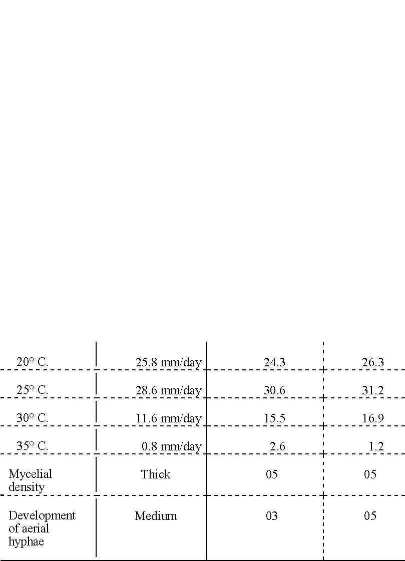

What is claimed is:

1. A new, distinct variety of Bunashimeji mushroom plant as substantially illustrated or described in the specification.
